(19) United States
(12) Patent Application Publication
(10) Pub. No.: US 2017/0031344 A1
(43) Pub. Date: Feb. 2, 2017

(54) METHOD OF MANAGING ADDITIVE APPLICATIONS IN AN AGRICULTURAL ENVIRONMENT

(71) Applicant: AGCO Corporation, Hesston, KS (US)

(72) Inventors: Jeffrey Michael Zimmerman, Lake Park, IA (US); John Peterson, Jackson, MN (US)

(21) Appl. No.: 15/301,537

(22) PCT Filed: Apr. 2, 2015
(86) PCT No.: PCT/US15/23991

§ 371 Date: Oct. 3, 2016

Related U.S. Application Data
(60) Provisional application No. 61/974,136, filed on Apr. 2, 2014.
(60) Provisional application No. 62/053,394, filed on Sep. 22, 2014.

(51) Int. Cl. G05B 19/406; G01N 33/00; A01C 21/00

(52) U.S. Cl. CPC G05B 19/406; A01C 21/007; G01N 33/0098; G05B 2219/32191

(57) ABSTRACT

In one embodiment, a method comprising regularly monitoring additive levels in plural areas of a field, wherein a first area of the plural areas comprises an additive rich area and a second area of the plural areas comprises an additive deficient area; performing statistical analysis on data corresponding to the monitored additive levels of the first and second areas; and determining when to apply additives to a third area of the plural areas of the field based on results of the statistical analysis.

### CROSS REFERENCE TO RELATED APPLICATION

This application claims the benefit of U.S. Provisional Application No. 61/974,136 filed Apr. 2, 2014, and 62/053,394, filed Sep. 22, 2014, both of which are hereby incorporated by reference in their entirety.

### TECHNICAL FIELD

The present disclosure is generally related to agricultural fertilizer applications.

### BACKGROUND

Agricultural production often requires the use of fertilizer or other additives to optimize plant growth and production. Fertilizers may provide various nutrients including nitrogen (N), phosphorus (P), potassium (K), calcium (Ca), magnesium (Mg), sulfur (S), boron (B), chlorine (Cl), copper (Cu), iron (Fe), manganese (Mn), molybdenum (Mo), zinc (Zn) and nickel (Ni).

Nitrogen is one example of a nutrient commonly found in fertilizers. All plants need nitrogen for healthy growth. It is an important component of many structural, genetic and metabolic compounds in plant cells, and it is a basic component of chlorophyll, the component by which plants use sunlight energy to produce sugars during the process of photosynthesis. Increasing the levels of nitrogen during the vegetative stage can strengthen and support plant roots, enabling plants to take in more water and nutrients. This allows a plant to grow more rapidly and produce large amounts of succulent, green foliage, which in turn can generate bigger yields, tastier vegetables, and a crop that is more resistant to pests, diseases, and other adverse conditions.

A nitrogen-deficient plant is generally small and develops slowly because it lacks the nitrogen it requires to manufacture adequate structural and genetic materials. Older leaves become yellow or pale green due to the lack of chlorophyll, beginning in the tips of the lower leaves and eventually spreading throughout the plant. In extreme deficiencies, the affected leaves become brownish, wither, die and hang down around the lower stem.

Too much nitrogen, however, can be as harmful to plants as nitrogen deficiency. For instance, slight over application may result in higher expenses for farmers and environmental damage. In extreme cases, when there are high levels of nitrogen present, plants may not produce flowers or fruit. As with nitrogen deficiency, the leaves may turn yellow and fall off the plant. Too much nitrogen can result in plant burning, which causes the plant to shrivel and die.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic diagram that illustrates an example computer network that may be used to implement an embodiment of an additive application system.

FIG. 2 is a schematic diagram that illustrates an example environment in which an embodiment of an additive application system may be used.

FIG. 3 is a schematic diagram that illustrates a data chart that may be created and used by an embodiment of an additive application system to perform statistical analysis of additive levels in a monitored area.

FIG. 4 is a block diagram of an embodiment of an example computing device used in an embodiment of an additive application system.

FIG. 5 is a flow diagram that illustrates an embodiment of an example additive application method.

### DESCRIPTION OF EXAMPLE EMBODIMENTS

### Overview

In one embodiment, a method comprising regularly monitoring additive levels in plural areas of a field, wherein a first area of the plural areas comprises an additive rich area and a second area of the plural areas comprises an additive deficient area; performing statistical analysis on data corresponding to the monitored additive levels of the first and second areas; and determining when to apply additives to a third area of the plural areas of the field based on results of the statistical analysis.

### DETAILED DESCRIPTION

Certain embodiments of an additive application system and method are disclosed that determine an optimal time to apply an additive to plants (e.g., crops) in a field. In one embodiment, an additive application system relies on timely and frequent measurements of additive levels corresponding to additive rich and additive deficient areas in the soil of one or more fields. The additive application system performs statistical analysis on the corresponding data from these respective sensed areas to determine when the plants begin to suffer due to a lack of the additive. In one embodiment, when the statistical analysis reveals that the additive deficient area of the field is being stressed by the additive deficiency, the additive application system can send a notification (e.g., alert) to the producer (e.g., farmer, farm operator, consultant, contractor, etc.) so that the producer can make preparations to treat the field. In some embodiments, the rate at which sensor readings change may be used to determine the severity of the deficiency and, related to that, to determine an urgency of the application. The additive application system enables the producer to apply the additive before the rest of the field suffers economic losses due to the additive deficiency, and in some embodiments, to prioritize the application of additives to the several fields based on the urgency of the nutrient (e.g., additive) requirement.

Digressing briefly, conventional systems that involve nitrogen sensing may rely on manually collecting data, or rely on a producer's best judgment (after laborious manual analysis) to determine when to apply the nitrogen to the field. Automation in the field of nitrogen treatment may contemplate rate selection (e.g., how much nitrogen to apply to a field) based on regional weather data (e.g., as opposed to sensed soil and/or sensed (e.g., via plant reflectance, etc.) plant conditions), but not when to apply the nitrogen. Further, some automated systems outline methods to calculate yield loss due to nitrogen deficiency, but again, not when to apply the nitrogen. In contrast, certain embodiments of an additive application system enable a more timely application of additives such as nitrogen by continuously performing a statistical analysis of sensed data and notifying the producer when it is time to apply the additive.

Having summarized certain features of an additive application system of the present disclosure, reference will now be made in detail to the description of the disclosure as illustrated in the drawings. While the disclosure will be described in connection with these drawings, there is no intent to limit it to the embodiment or embodiments disclosed herein. For instance, though emphasis is placed on nitrogen as the additive of focus, applications involving other additives (e.g., phosphorus (P), potassium (K), calcium (Ca), magnesium (Mg), sulfur (S), boron (B), chlorine (Cl), copper (Cu), iron (Fe), manganese (Mn), molybdenum (Mo), zinc (Zn) and nickel (Ni)) may likewise be used, and hence are contemplated to be within the scope of the disclosure. Further, although the description identifies or describes specifics of one or more embodiments, such specifics are not necessarily part of every embodiment, nor are all of any various stated advantages necessarily associated with a single embodiment. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

Note that in some embodiments, reference hereinafter made to a nitrogen rich area includes an area where there is reasonable certainty that the crop is not limited by the availability of nitrogen. In some embodiments, a nitrogen rich area is a defined area characterized by having an average or median nitrogen concentration greater than a threshold concentration of nitrogen. It should be appreciated by one having ordinary skill in the art that such a threshold may vary depending on one or more factors, such as crop type, the crop growth stage, soil type, soil temperature, soil moisture level, among other factors. Detection of the nitrogen concentration primarily, if not entirely, relies on plant reflectance (as opposed to soil testing, though soil testing may be used as at least a supplemental method of detection in some embodiments). In some embodiments, the application rate of nitrogen is above an economic threshold by a multiple of natural soil variability (e.g., two (2) standard deviations), which helps to ensure that, where there is a natural deficiency of nitrogen, the plant is not nitrogen deficient. In some embodiments, reference hereinafter made to a nitrogen deficient area includes areas where there is reasonable certainty that the crop will experience nitrogen stress prior to the rest of the field. In some embodiments, a nitrogen deficient area is a defined area characterized by having an average or median nitrogen concentration less than a threshold concentration of nitrogen.

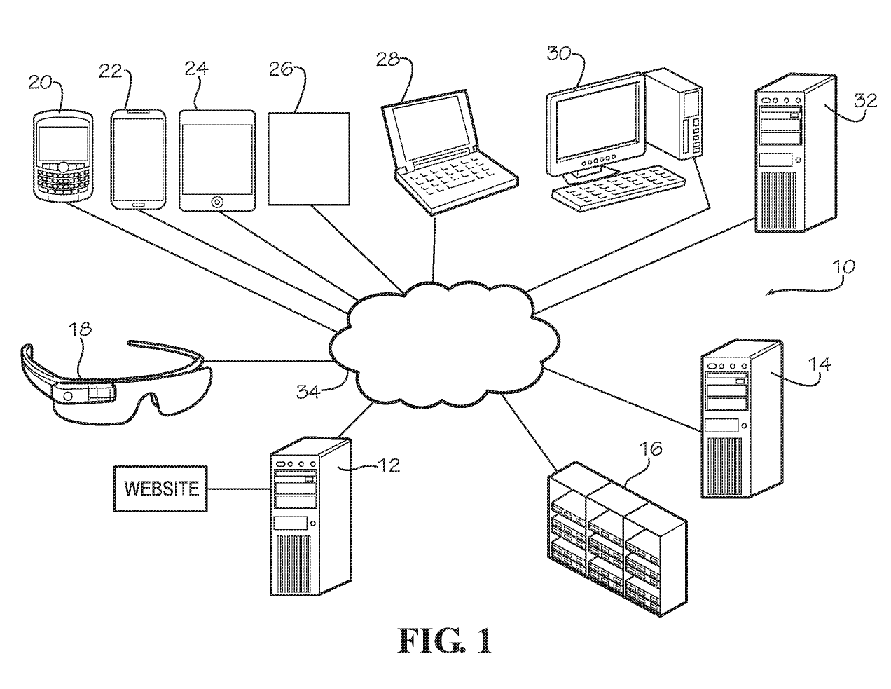

Reference is made to FIG. 1, which is a schematic diagram that illustrates an example computer network 10 that may be used to implement an embodiment of an additive application system. One having ordinary skill in the art should appreciate in the context of the present disclosure that the example computer network 10 depicted in FIG. 1 is merely illustrative, and that other networks with like functionality may be used in some embodiments. In one embodiment, one or more of the functions of the additive application system may be implemented with a computer program or programs that operate in conjunction with computer and communications equipment broadly referred to by the computer network 10 in FIG. 1. The example computer network 10 may include one or more host computers or systems 12, 14, 16 (hereinafter referred to simply as “host computers”) and a plurality of electronic or computing devices 18, 20, 22, 24, 26, 28, 30, 32 that may access the host computers via a communications network 34. The host computers 12, 14, 16 may serve as repositories for data and programs (e.g., executable code) used to implement certain functions of the additive application system as described in more detail below. The host computers 12, 14, 16 may be any computing and/or data storage devices such as network or server computers and may be connected to a firewall to prevent tampering with information stored on, or accessible by, the computers. One of the host computers, such as host computer 12, may be a device that operates or hosts a website accessible by at least some of the devices 18-32. The host computer 12 may include conventional web hosting operating software and an Internet connection, and is assigned a Uniform Resource Locator (URL) and corresponding domain name so that the website hosted thereon can be accessed via the Internet in a conventional manner. One or more of the host computers 12, 14, 16 may host and support a database or other data structure for storing Global Navigation Satellite System (GNSS) information, as explained below. The database may be accessible, for example, via the website operated by the host computer 12. Although three (3) host computers 12, 14, 16 are described and illustrated herein, certain embodiments of the additive application system may use any combination of host computers and/or other computers or equipment. For example, the computer-implemented features and services described herein may be divided between the host computers 12, 14, 16 or may all be implemented with only one of the host computers. Furthermore, the functionality of the host computers 12, 14, 16 may be distributed amongst many different computers in a cloud computing environment, including the other devices 18-32 in some embodiments.

The electronic devices 18-32 may include various types of devices that can access the host computers 12, 14, 16 via the communications network 34. By way of example, the electronic devices 18-32 may include one or more laptop, personal or network computers 28-32 as well as one or more smart phones, tablet computing devices or other handheld, wearable and/or personal computing devices 18-24. The devices 18-32 may include one or more devices or systems 26 embedded in, or otherwise associated with, a machine wherein the device or system 26 enables the machine, an operator of the machine, or both to access one or more of the host computers 12, 14, 16. Each of the electronic devices 18-32 may include or be able to access a web browser and a conventional Internet connection such as a wired or wireless data connection. As explained below, the device 26 may be associated with a position determining system or device on a mobile machine and may be operable to communicate with one or more of the host computers 12, 14 or 16 to receive information necessary for the position determining system or device to connect with or otherwise access a GNSS data source.

The communications network 34 preferably is, or includes, the Internet, but may also include other communications networks such as a local area network, a wide area network, a wireless network, or an intranet. For instance, the network 34 may comprise one or more networks, including a wireless network (e.g., cellular, WiFi, Wide Area Network, Local Area Network, radio frequency, terrestrial, satellite, etc.) and a wired network (e.g., POTS, cable, etc.), as should be appreciated by one having ordinary skill in the art. For example, the electronic devices 18-32 may wirelessly communicate with a computer or hub in a facility via a local area network (e.g., a Wi-Fi network), which in turn communicates with one or more of the host computers 12, 14, 16 via the Internet or other communication network. Other components and/or facilities known in the art and which may be used in some embodiments, such as cellular towers, DSLAMs, ISP facilities, etc., are omitted here for brevity.

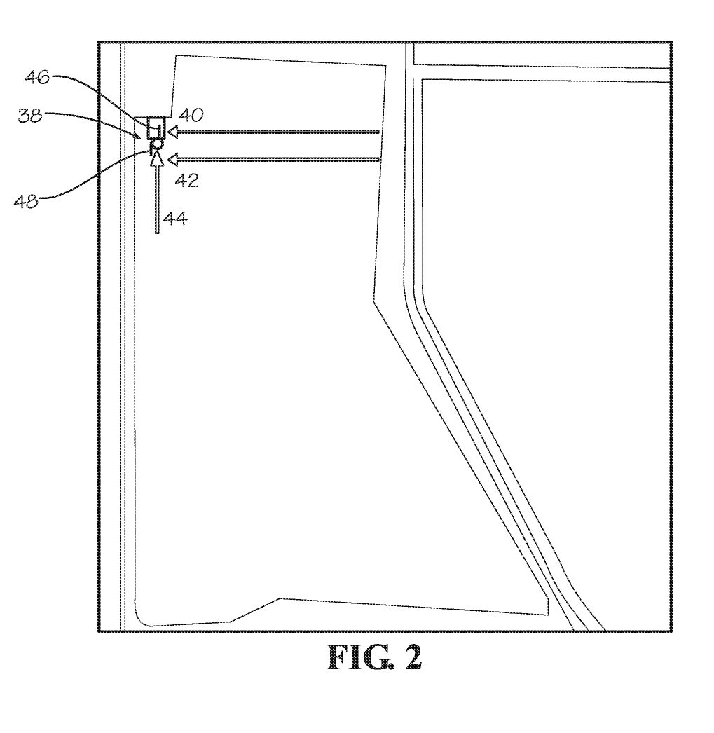

Reference is now made to FIG. 2, which illustrates an example environment 36 in which an embodiment of an additive application system may be used. In this example, the additive application system is used to facilitate management of nitrogen application (e.g., timing and optionally prioritization) in an agricultural environment 36, though as indicated above, some embodiments may manage other additives. The example environment 36 of FIG. 2 depicts plural fields, where one of the fields comprises a test area or section 38 to be used as a representative area for purposes of monitoring and deploying statistical data analytics to determine a critical time or an optimal time to apply the nitrogen. It should be appreciated that the use of a test area 38 in FIG. 2 is merely illustrative, and that the entire field or plural portions thereof (or plural fields or portions thereof) may be used for purposes of monitoring and applying statistical data analytics in some embodiments. A first portion 40 of a crop area is managed to be rich in nitrogen, while a second portion 42 of the crop area is managed to be nitrogen deficient (e.g., the crop will experience nitrogen stress prior to the rest of the crop area). As illustrated in FIG. 2, the first and second areas 40, 42 may be a relatively small portion of the overall crop area (and it should be appreciated that in some embodiments, a farmer may choose to minimize the nitrogen deficient area 42 relative to the nitrogen rich area 40), or in some embodiments, may encompass a greater overall crop area.

One or more sensors may be used to collect data from the first and second portions 40, 42 of the crop area indicative of nitrogen levels. In this example, a ground station 44 comprising two (2) Difference Vegetation Index (NDVI) sensors 46, 48 is located between the two areas 40, 42. Each NDVI sensor 46 and 48 is positioned to monitor the respective nitrogen rich area 40 and nitrogen deficient area 42. The NDVI is a simple graphical indicator that can be used to analyze remote sensing measurements, typically but not necessarily from a space platform, and assess whether the target being observed contains live green vegetation or not. Note that although NDVI represents one metric or index related to plant nitrogen demand, other metrics or data related to plant nitrogen demand may be used, include Red Edge or Near Infrared (NIR) NVDI, chlorophyll index, among others metrics well-known to those having ordinary skill in the art. Also, though monitoring is illustrated in FIG. 2 using stationary ground sensors 46, 48 (e.g., measuring plant reflectance), it should be appreciated by one having ordinary skill in the art that fewer or greater quantities of sensors may be used in some embodiments. Also, various types of methods and/or sensors may be used to collect the data indicative of nitrogen levels or nitrogen health in the crop. For instance, the data may be collected from sensors 46, 48 in or proximate to the crop area, as illustrated in FIG. 1, or may be collected from remote sensors such as sensors mounted on aerial vehicles or satellites. Stated otherwise, remote sensing may be used (e.g., space deployed sensors, such as one or more satellite-based sensors or airplane or drone-mounted sensor(s)), or mobile sensors in addition to satellite or aerial sensors, such as hand-held sensors, ground vehicle mounted sensors, or any combination thereof. Note that the required or defined size of each respective area 40, 42 depends on the mechanism for monitoring the area. For instance, satellite-based monitoring may require that each respective area be the size of at least one pixel of remote imagery data.

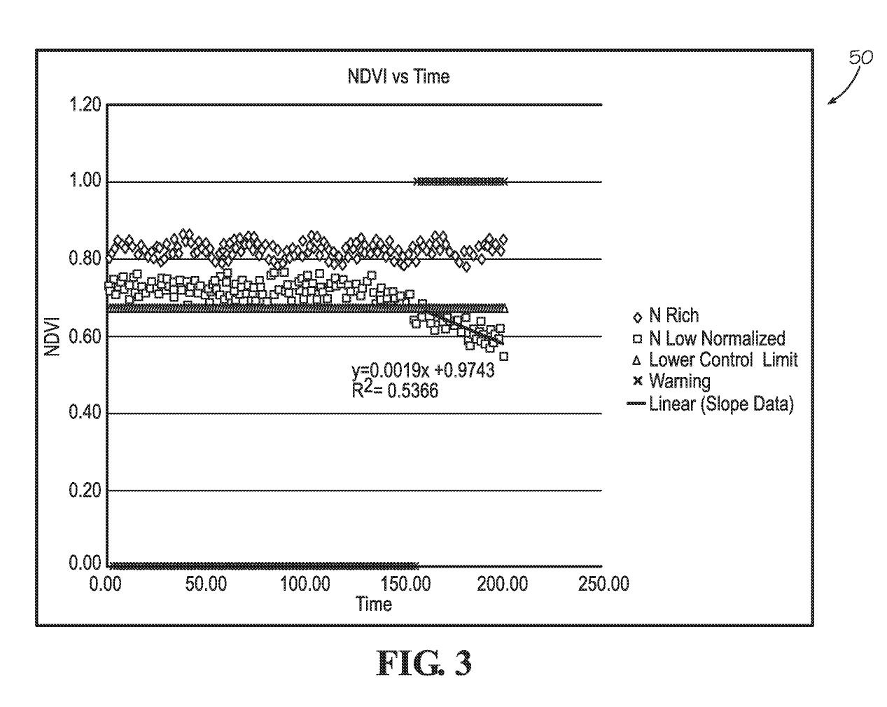

In one example operation, the additive application system monitors the areas 40, 42 using the sensors 46, 48 of the ground station 44, and applies statistical analysis to the representative sensor data to determine when the rest of the crop area may suffer from nitrogen stress. Nitrogen may then be applied to the rest of the crop area to avoid or minimize nitrogen stress. Thus, using the additive application system, a producer may anticipate when nitrogen stress may occur in the crop area generally and apply nitrogen at the optimal time. Once the data is collected, any of various methods may be used to assess the need for nitrogen application in the crop area. Referring to FIG. 3, shown is a schematic diagram that illustrates information that may be used by an embodiment of an additive application system to perform statistical analysis of additive levels in a monitored area. In this example, a data chart 50 is depicted, with the horizontal axis providing a time axis and the vertical axis providing a monitored metric, such as NDVI. The top group of data points 52 correspond to NDVI values for the nitrogen rich area 40, and the lower group of data points 54 correspond to NDVI values for nitrogen deficient area 42. In one embodiment, a computing device or devices (e.g., of the computer network 10 of FIG. 1) of the additive application system, either local to the ground station 44 or remotely located (e.g., via input according to telemetry communication from the sensors 46, 48 or an associated device), performs statistical analysis on the data points 52, 54. For instance, statistical analysis may involve trend analysis, where nitrogen rich NDVI (data points 52) may be used to normalize nitrogen deficient NDVI to correct for non-nitrogen fluctuations, resulting in the normalized data points 54. In this example, the cyclical or sine-like fluctuation is due to daily variation, where the distance between each peak of the data points 52 corresponds to a single-day interval of recorded data. For instance, the nitrogen deficient data is normalized using the variation of the nitrogen rich data points 52 from the nitrogen rich mean (statistical) value. Note that although plural readings are shown taken on a daily basis in FIG. 3, a different temporal frequency for readings may be used, including more frequently or less frequently.

Also, the additive application system, as part of the statistical analysis, provides a lower control limit 56. The lower control limit 56 serves as a lower limit or threshold, below which triggers an alert to a producer. More particularly, the lower control limit 56 is established based on a statistical analysis of the (e.g., normalized) NDVI deficient data 54. In one embodiment, when a statistical value (e.g., the average) of a defined quantity (e.g., four (4)) of NDVI deficient data points 54 is below the lower control limit 56, the additive application system provides an alert or warning to a producer to warn him or her that the field requires fertilization. That is, the alert is indicating to the producer when application is required. Such an alert may be provided in response to the determination based on different statistical values (e.g., median) and/or according to a different quantity of values below the control limit 56 (e.g., fewer or greater). In some embodiments, the alert may not be responsive to the determination, but rather, based on a periodic status update. In one embodiment, the notification may be achieved via a wireless communication to a portable electronic device (e.g., phone, laptop, etc.). The format of the message may be via voice message, text (e.g., SMS), among others well-known to those having ordinary skill in the art. In some embodiments, the notification may be delivered to a workstation, or to an operator's console in a machine. For instance, the producer may log on to email or to a web-site via browser software, and observe an alert that is presented visually on the website (e.g., after logging into his or her account). In some embodiments, a combination of these methods may be deployed, where a cryptic alert is provided to a portable device with a link to a website that details the circumstances or area surrounding the alert, such as the area identification, extent of deficiency, etc.

In some embodiments, the additive application system may provide further information. For instance, in situations where there are plural monitored fields of respective nitrogen rich and nitrogen deficient areas (in addition to the rest of the fields), the additive application system may order the fields or areas by increasing or decreasing nitrogen deficiency, enabling a prioritization of the urgency of treatment. In other words, the producer may be provided with a list of fields or areas in descending order of treatment needs, where the area in most need of treatment (e.g., fertilization) is listed at the top or otherwise highlighted as being closest in time to realizing economic loss due to nitrogen deficiency, the second most in need of treatment listed second in the list, and so on. In one embodiment, the urgency or severity of the nitrogen deficiency is determined based on a rate of change of the nitrogen levels (e.g., a slope 58 of the data points located at or beneath the lower control limit 56). Slopes of greater magnitude indicate a greater nitrogen deficiency and contribute to a greater urgency of application or treatment.

It should be appreciated by one having ordinary skill in the art, in the context of the present disclosure, that there exists a variety of mechanisms by which certain embodiments of additive application systems may operate. For instance, for ground station-based sensing, the ground station 44 may comprise a host computer 12, 14, and/or 16 coupled to the sensors 46, 48, which receives and processes the sensor data (e.g., performs statistical analysis). The host computer 12, 14, and/or 16 may comprise transceiver functionality (e.g., equipped with, or coupled to, a radio and/or cellular modem), and may be configured to communicate the alerts and/or other information (e.g., a visual representation of the data chart) to a communication-capable device 18-32 (e.g., smartphone, laptop, workstation, operator console, etc.) associated with the producer. In some embodiments, the ground station 44 may be equipped with transceiver functionality that communicates the sensor data over one or more networks to a host computer 12, 14, and/or 16, where the processing (e.g., statistical analysis) is performed remote from the field, such as at a facility (e.g., farm, office, etc.) associated with the producer. In the latter embodiment, the sensor data may be communicated to the host computer 12, 14, and/or 16 via an intermediate device, such as one of the devices 18-32, which in turn uploads the sensor data to the host computer 12, 14, and/or 16 for processing. In some embodiments, such as where remote sensing is deployed, the sensor data may be communicated through one or more networks to a host computer 12, 14, and/or 16 proximal to, or remote from, the producer. The host computer 12, 14, and/or 16 processes the data and communicates the alerts and/or other information (e.g., a visual representation of the data chart 50) to the producer over a wired and/or wireless medium. It should be appreciated that, in the examples listed above, the host computer 12, 14, and/or 16 is described as the location for processing and receiving of sensor data, though in some embodiments, host functionality may reside in one or more of the other devices 18-32.

As indicated above, the data may be analyzed by a computing device, such as a computing device associated with a tractor or other mobile machine, or a remote computing device such as one of the computers 12, 14, 16 described above and illustrated in FIG. 1. In some embodiments, the analysis may be performed at the ground station 44 and communicated (e.g., via a wireless communication, such as telemetry), or as uploaded through the use of memory stick or otherwise that is manually retrieved at the ground station 44. The additive application system may include one or more computer programs or devices configured to communicate a message to a user (e.g., producer) alerting the user to the impending need for additives to be applied to an agricultural area. The message may be communicated via one of the devices 18-24, 28-32 described above and illustrated in FIG. 1, for example. In response to the alert, the producer treats the nitrogen deficient plants. The additive application system may present to the producer (e.g., as part of the alert, or as a link associated with the alert for subsequent access) an approximate schedule or timeline of when the treatment is required, prioritized by urgency, or in some embodiments, an approximate due date beyond which economic loss is likely. In some embodiments, the presence of the alert has a built in timeline, such that the producer knows when he or she receives the alert, it is a matter of days or other time span before economic loss is likely.

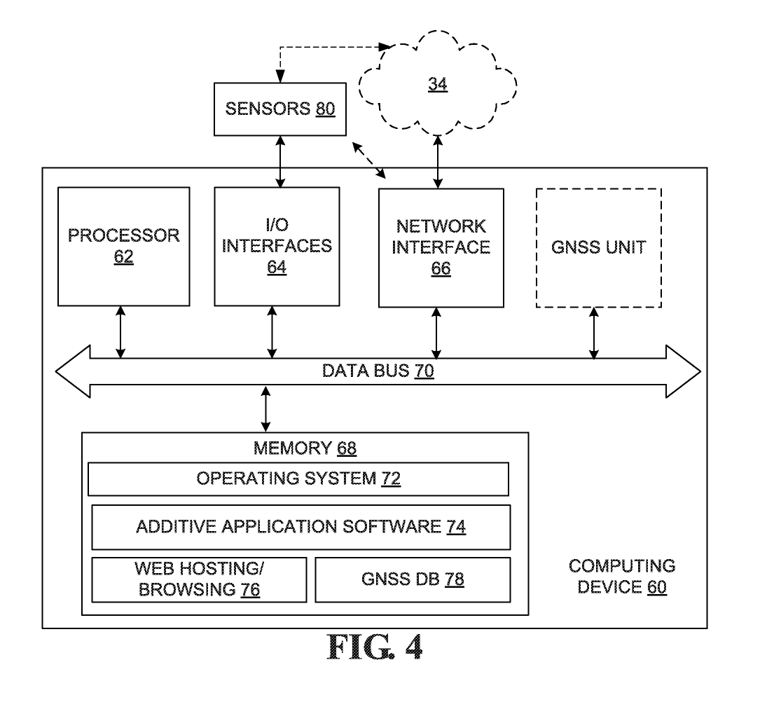

FIG. 4 illustrates an example embodiment of a computing device 60. The computing device 60 may comprise any one of the devices 12-32 depicted in FIG. 1 that is configured to perform functionality associated with certain embodiments of the additive application system. One having ordinary skill in the art should appreciate in the context of the present disclosure that the example computing device 60 is merely illustrative, and that some embodiments of computing devices may comprise fewer or additional components, and/or some of the functionality associated with the various components depicted in FIG. 4 may be combined, or further distributed among additional modules, in some embodiments. It should be appreciated that the computing device 60 may reside in a machine (e.g., tractor, combine harvester, etc.), at a ground station 44 (FIG. 2), at a system or device proximal to a producer (e.g., at a facility, such as farm or office or wherever the producer is located) or remote from a producer (e.g., at a service provider facility, etc.), or with functionality distributed among two or more of these locations. In FIG. 4, the computing device 60 is depicted in this example as a computer system, but all or a portion of the additive application system functionality may be embodied in a programmable logic controller (PLC), field programmable gate array (FPGA), application specific integrated circuit (ASIC), among other devices. As is known, the functionality of certain embodiments of the additive application system, when carried out in an ASIC or FPGA, is designed into the ASIC or FPGA according to a hardware description language (e.g., Verilog, VHDL, etc.). For embodiments using an FPGA, separate logic blocks (e.g., combinational logic or sub-portions thereof (e.g., simple logic gates, such as AND, OR gates)) may be used for separate or combined algorithmic steps of an additive application method. Programming of a PLC to perform one or more functionality of the additive application system may be achieved using any of a variety of known mechanisms, such as via application software on a personal computer and communication with the PLC over a suitable connection (e.g., Ethernet, cabling according to RS-232, RS-485, etc.) to enter or edit ladder-type logic as is known, or via a programming board interface for storage of the program into memory (e.g., EEPROM, etc.). It should be appreciated that certain well-known components of computer systems are omitted here to avoid obfuscating relevant features of the computing device 60. In one embodiment, the computing device 60 comprises one or more processors, such as processor 62, input/output (I/O) interface(s) 64, a network interface 66, and a memory 68, all coupled to one or more data busses, such as data bus 70.

The memory 68 may include any one or a combination of volatile memory elements (e.g., random-access memory RAM, such as DRAM, and SRAM, etc.) and nonvolatile memory elements (e.g., ROM, hard drive, tape, CDROM, etc.). The memory 68 may store a native operating system, one or more native applications, emulation systems, or emulated applications for any of a variety of operating systems and/or emulated hardware platforms, emulated operating systems, etc. In the embodiment depicted in FIG. 4, the memory 68 comprises an operating system 72 and additive application software 74. In some embodiments, the memory 68 further comprises browser or web-hosting software 76 (depending on the role of the computing device 60), and GNSS database 78. It should be appreciated that in some embodiments, additional or fewer software modules (e.g., combined functionality) may be deployed in the memory 68 or additional memory. In some embodiments, a separate storage device may be coupled to the data bus 70, such as a persistent memory (e.g., optical, magnetic, and/or semiconductor memory and associated drives). The storage device may be a removable device, such as a memory stick or disc.

In one embodiment, the executable code of the additive application software 74 is executed by the processor 62 to carry out the functionality of the additive application system. For instance, the additive application software 74 receives sensor input from sensors 80. Sensors 80 may comprise ground sensors, or mobile sensors in proximity to the computing device 60. In some embodiments, the sensors 80 may include remote sensors (e.g., satellite or aerial-based), which may be received via the network interface 66 from over the network 34. In some embodiments, the computing device 60 may be equipped with, or directly coupled to, a GNSS receiver that receives satellite sensor data, and/or the aerial sensor data may be directly received via transceiver functionality associated with the network interface 66. The sensor data may be associated with time and position data (through coupling or association with a GNSS receiver or GNSS receiver data), which the additive application software 74 uses in conjunction with GNSS field maps loaded into and stored in GNSS database 78 to identify the locations of the nitrogen deficient and nitrogen rich areas. For instance, in a ground station application, the sensors 46, 48 (FIG. 2) may be associated with known and fixed position data (or in some embodiments, sensed position data when associated with a coupled GNSS receiver), which the ground station 44 (FIG. 2) may include as packet information when communicating the sensor data to the computing device 60. The additive application software 74 records the data, and performs statistical analysis on the same to determine trends and normalize the data, as well as to determine when to send an alert (e.g., based on the lower control limit 56) and to determine priority (e.g., slope determinations) in the case where several areas are monitored. The additive application software 74 further sends alerts, formatted as required for communication over the network interface 66, to indicate there is a nitrogen-stressed area and advise the producer when (e.g., immediately or according to a schedule, among other mechanisms for advising of when to apply the nitrogen) to apply the nitrogen to the nitrogen deficient area or areas. In some embodiments, the additive application software 74 works in conjunction with web-hosting services 76 to provide a link for further detail of any detected deficiencies that a producer can access via browser software on his or her electronic device. The additive application software 74 may provide, in some embodiments, additional information such as the data chart 50 or other information, such as the required nitrogen dosage and/or rate to remedy the deficiency. The alert information provided by the additive application software 74 may be presented on an interface to a producer (e.g., visual and/or audio interface, such as a voice recording conveyed by speakers on a smartphone or visual presentation on a phone or laptop).

Execution of the additive application software 74 may be implemented by the processor 62 under the management and/or control of the operating system 72. For instance, as is known, the source statements that embody the method steps or algorithms of the additive application software 74 may be translated by one or more compilers of the operating system 72 to assembly language and then further translated to a corresponding machine code that the processor 62 executes to achieve the functionality of the additive application software 74. Variations of this execution process are known, depending on the programming language of the software. For instance, if Java-based, the compiled output may comprise bytecode that may be run on any computer system platform for which a Java virtual machine or bytecode interpreter is provided to convert the bytecode into instructions that can be executed by the processor 62. Also, register transfer language (or other hardware description language) may be used to translate source code to assembly language, which the one or more operating system compilers translate to executable machine code. The processor 62 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 60.

The I/O interfaces 64 provide one or more interfaces to the sensors 80, which may include ground and/or mobile sensors (and in some embodiments, remote sensors). The I/O interfaces 64 may further be configured for the receipt of input corresponding to user interfaces, such as a keyboard, microphone, mouse, touch-screen, as well as for output to display devices (e.g., flat panel display screen, LCD display, etc.).

The network interface 66 comprises any number of interfaces for the input and output of signals (e.g., analog or digital data) for conveyance of information (e.g., data) over the network 34. The input may comprise sensor data from remote sensors 80 (e.g., GNSS-based or aerial-based sensor data), and the output may comprise alerts that advise or indicate to a producer when nitrogen (or other additives) need to be applied, as well as the communication of other information (e.g., data chart 50, nitrogen application rate, dosage, recommended application schedule, priority list, etc.). The network interface 66 may include transceiver functionality, such as a radio modem.

When certain embodiments of the computing device 60 are implemented at least in part with software (including firmware), as depicted in FIG. 4, it should be noted that the software can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 60 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Note that the computing device 60 may communicate among other components according to ISO-bus in some embodiments.

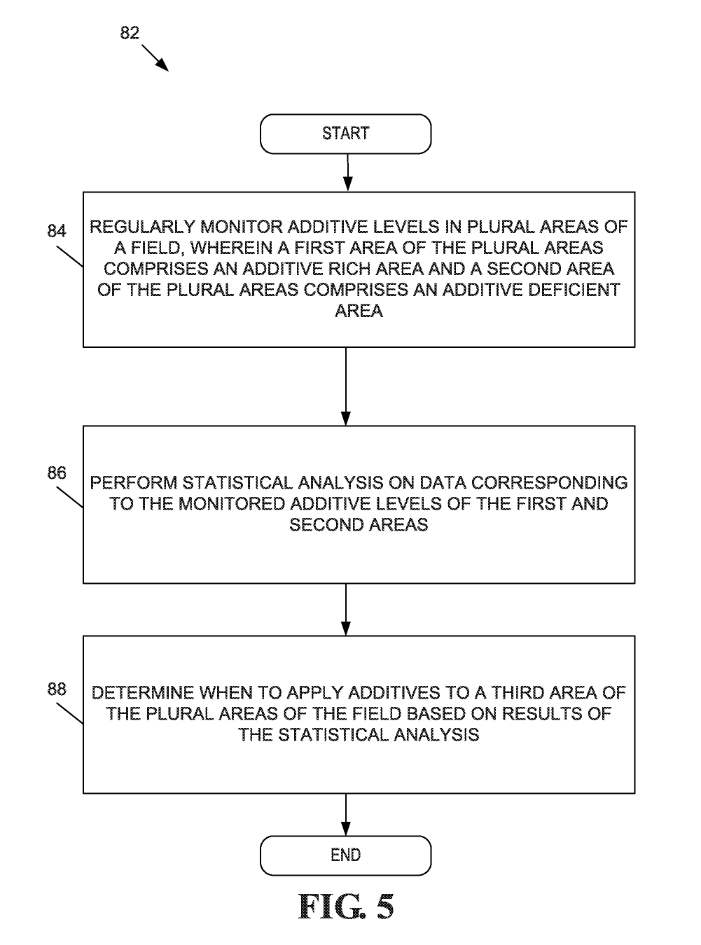

In view of the above description, it should be appreciated that one embodiment of an additive application method 82, depicted in FIG. 5, which in one embodiment may be performed by the computing device 60, comprises regularly monitoring additive levels in plural areas of a field, wherein a first area of the plural areas comprises an additive rich area and a second area of the plural areas comprises an additive deficient area (84). For instance, regularly monitoring may involve multiple readings through a day, or over a course of a multitude of days (e.g., once daily, once every other day, etc.). The method 82 further comprises performing statistical analysis on data corresponding to the monitored additive levels of the first and second areas (86). For instance, the data may include a metric such as NVDI, among other data relevant to plant demand for nitrogen (or other additives). The method 82 further comprises determining when to apply additives to a third area of the plural areas of the field based on results of the statistical analysis (88). The third area may comprise areas outside of a control area, or areas of the field that include all or a portion of the first and/or second areas. The determination may be performed as regularly as the data recordings, or periodically or aperiodically, or upon producer demand (e.g., input soliciting the determination).

Any process descriptions or blocks in flow diagrams should be understood as representing modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein. Although the disclosed systems and methods have been described with reference to the example embodiments illustrated in the attached drawing figures, it is noted that equivalents may be employed and substitutions made herein without departing from the scope of the disclosure as protected by the following claims.

At least the following is claimed:

1. A method, comprising:
regularly monitoring additive levels in plural areas of a field, wherein a first area of the plural areas comprises an additive rich area and a second area of the plural areas comprises an additive deficient area;
performing statistical analysis on data corresponding to the monitored additive levels of the first and second areas; and
determining when to apply additives to a third area of the plural areas of the field based on results of the statistical analysis.

2. The method of claim 1, further comprising applying the additives to the additive deficient area based on the determination.

3. The method of claim 1, further comprising notifying a producer of the determination by providing a wireless communication to a portable electronic device.

4. The method of claim 3, wherein the wireless communication comprises one or a combination of an automated text message or automated voice message.

5. The method of claim 1, wherein the third area comprises an area outside of the first and second areas or areas that overlap one or a combination of the first or second areas.

6. The method of claim 1, further comprising notifying a producer of the determination by alerting the producer on an interface of a computing device.

7. The method of claim 6, wherein the interface comprises a web-interface.

8. The method of claim 1, wherein monitoring comprises remote sensing.

9. The method of claim 1, wherein monitoring comprises using ground sensors.

10. The method of claim 1, wherein monitoring comprises mobile sensing.

11. The method of claim 1, wherein regularly monitoring comprises daily monitoring.

12. The method of claim 1, wherein the data comprises a metric corresponding to plant demand for the additive.

13. The method of claim 12, wherein the additive comprises nitrogen.

14. The method of claim 13, wherein the metric comprises Normalized Difference Vegetation Index (NDVI).

15. The method of claim 1, wherein performing statistical analysis comprises establishing a lower control limit, wherein the determination is based on a statistical value that falls below the lower control limit.

16. The method of claim 15, wherein performing statistical analysis further comprises using data corresponding to the first area to normalize data corresponding to the second area.

17. The method of claim 15, further comprising monitoring one or more respective additional additive rich and additive deficient areas.

18. The method of claim 17, wherein performing statistical analysis further comprises:
determining a slope corresponding to data values involved in the determination of the statistical value; and
prioritizing an order of applying the additive to the additive deficient area and the one or more additional additive deficient areas based on the slope determination.

19. A system, comprising:
one or more sensors that detect nitrogen levels in nitrogen rich and nitrogen deficient areas of a field; and
a computing device configured to:
receive data from the one or more sensors corresponding to plant demand for the nitrogen rich and nitrogen deficient areas;
perform statistical analysis on the data; and
determine when to apply nitrogen to the field based on results of the statistical analysis.

20. A computing device, comprising:
a memory comprising executable code; and
a processor configured by the executable code to:
receive data from one or more sensors, the data corresponding to plant demand for nitrogen rich and nitrogen deficient areas of a field;
perform statistical analysis on the data;
determine when to apply nitrogen to the nitrogen deficient area based on results of the statistical analysis; and
provide an alert to a producer responsive to the determination, the alert indicating a need to apply the nitrogen to the field.
